(19) United States
(12) Patent Application Publication
(10) Pub. No.: US 2003/0018386 A1
(43) Pub. Date: Jan. 23, 2003

(54) Anterior chamber angle-supported intraocular lenses with flexible optic and rigid fixation members

(75) Inventors: Stephen W. Laguette, Laguna Niguel, CA (US); Robert T. Nguyen, Irvine, CA (US)

Correspondence Address: Frank J. Uxa, Stout, Uxa, Buyan & Mullins, LLP, Suite 300, 4 Venture, Irvine, CA, 92618, US

(73) Assignee: Allergan Sales, Inc., Irvine, CA

(21) Appl. No.: 09/908,515

(22) Filed: Jul. 18, 2001

(51) Int. Cl. A61F 2/16

(52) U.S. Cl. 623/6.46; 623/6.49

(57) ABSTRACT

An intraocular lens having an optic portion and a plurality of fixation members coupled thereto. The optic portion includes an optic and a pair of outwardly extending flanges. The fixation members are relatively more rigid than the optic and attach to the flanges. The flanges provide lines of force transmission between pairs of fixation members so that compressive forces are not transmitted through the optic, therefore reducing vaulting. The fixation members may be bonded or mated to the flanges. A bridge between two opposed fixation members may be provided to form a fixation system, the bridges being attached to the flanges.

### BACKGROUND OF THE INVENTION

This invention relates to intraocular lenses (IOLs). More particularly, the invention relates to foldable IOLs placed in the anterior chambers of eyes which provide proper IOL retention forces and a reduction in the tendency of the optics of the IOLs to vault.

Intraocular lenses (IOLs) are commonly used to modify or enhance vision. IOLs can be placed at various positions or locations within the eye. For example, IOLs can be placed in the anterior chamber (AC) of the eye, that is, the region of the eye posterior of the cornea and anterior of the iris.

IOLs may generally be classed by material. Hard or rigid IOLs are distinguished from soft IOLs that may be folded to facilitate implantation through a small incision in the cornea (and capsular bag for posterior lenses).

Although there are substantial advantages to placing the IOL in the anterior chamber of the eye, various complications have been reported as a result of the presence of IOLs in such anterior chambers. Among other problems, anterior chamber IOLs have been reported to cause decentration or offsetting displacement away from a preferred optical axis.

IOLs advantageously have been foldable for insertion through small incisions in eyes, particularly for insertion in the capsular bags in the posterior chambers of the eyes.

When implanted, both soft and rigid anterior chamber IOLs exert retention forces on their outer ends; that is, the IOL is typically slightly oversized relative to the peripheral anterior chamber structure. A slight amount of such retention forces is desirable so that the lens is held in place or centered, otherwise a loose fit might cause vision and other problems.

As stated, anterior chamber IOLs may be oversized and flexible in the plane of the IOL such that they are placed in compression when implanted. However, a balance must be observed between sufficient compression for a good fit and unwanted endothelial cell loss and pupil ovalling from excessive compression. In addition, the problems of corneal touch and further endothelial cell loss may arise in some current IOLs, whether formed of soft or rigid materials, which may deflect along the optical axis even with only a small magnitude of compressive fit. As a result, IOL manufacturers must provide a range of sizes to fit the IOL to a particular patient's eye and reduce, or even substantially minimize, retention forces and the potential for axial deflection.

A common technique for placement of an intraocular lens in the anterior chamber is within the iridio-corneal angle, in a so-called “angle-supported” configuration. A number of non-foldable angle-supported anterior chamber intraocular lenses are fabricated from rigid materials, such as polymethyl methacrylate (PMMA). These rigid anterior chamber intraocular lenses are typically based upon a Kelman design of thin, flexible haptics with 3 or 4 footplates.

Small incision surgery requires a foldable anterior chamber intraocular lens. Recent attempts have been made to create a foldable anterior chamber intraocular lens with a flexible optic and rigid PMMA haptic/footplate materials for stable fixation. Unfortunately, these designs provide less than desirable foldability, and do not to lend themselves to insertion through an instrument similar to a Bartell-style inserter, for example, the IOL inserter as described in Bartell U.S. Pat. No. 4,681,102, the disclosure of which is hereby incorporated herein in its entirety by reference.

Other recent attempts of fabricating a one-piece intraocular lens from flexible material have produced designs with minimal compressive retention forces. As result, these designs may be less stable in the iridio-corneal angle and may allow unwanted intraocular lens movement.

It would be advantageous to provide soft anterior chamber IOLs which provide one or more of the following: reduced incidences of one or more known complications caused by prior anterior chamber IOLs, effective and safe folding for insertion in the eye, safe and effective fit to a range of sizes of eyes, a minimum of translational movement of the optic of the IOL along the optical axis from the compressive fit in the eye, and an otherwise stable optic to avoid unwanted movement.

### SUMMARY OF THE INVENTION

New IOLs for implantation in eyes, in particular in anterior chambers of the eyes, have been discovered. The present IOLs, are sized and structured to reduce the incidence of one or more known complications in the eye caused by prior anterior chamber IOLs.

In one aspect of the invention, a foldable intraocular lens for implantation in the anterior chamber of an eye is provided. The intraocular lens comprises an optic centered on an optical axis, at least two pairs of fixation members each having a proximal end and a distal end adapted to engage the anterior chamber of an eye, and at least two optic/fixation member interface portions to which the optic and the proximal ends of the fixation members attach. The interface portions each provide a line of compressive force transmission that does not intersect the optic between the proximal ends of each pair of fixation members.

In a preferred embodiment, the optic is circular and the interface portions extend outward from opposite sides of the optic to form planar flanges. Each flange may be defined by two side edges that project generally tangentially from the circular optic and converge toward each other in an outward direction from the optic, and an outer straight edge. The line of compressive force transmission between the proximal ends of each pair of fixation members desirably extend parallel to and adjacent the outer straight edge of the respective flange. Further, the proximal ends of each pair of fixation members may be co-linear. Each fixation member may include a central portion that converges inward toward a second fixation member, wherein the distal end of each fixation member terminates in a footplate that is wider than the central portion.

Another embodiment of the invention is a foldable intraocular lens for implantation in the anterior chamber of an eye, comprising:

an optic centered on an optical axis;

a pair of planar flanges extending in opposite directions from the optic; and

two pairs of fixation members each having a proximal end attached to one of flanges and a distal end adapted to engage the anterior chamber of an eye, the attachment of each pair of fixation members to the flanges providing a line of compressive force transmission that does not intersect the optic between the proximal ends of the two fixation members in the pair.

Each pair of fixation members may be formed of a single element connected by a bridge that attaches to the flange. Where the optic is circular, each flange is defined by two side edges that project generally tangentially from the circular optic and converge toward each other in an outward direction from the optic, and an outer straight edge. Each bridge desirably attaches to the outer straight edge of the respective flange. Each bridge may include a groove into which the outer straight edge of the respective flange inserts. The outer straight edge of each flange inserts into the groove of the respective bridge a depth that places an inner edge of the bridge generally on a tangent to the circular optic. The inner edge of each bridge includes a central corner that is recessed away from the optical axis and pair of half-edges diverging outwardly from the central corner and angled generally in the direction of the optic.

In one embodiment, all four fixation members are identical in configuration. In another embodiment, a first one of each pair of fixation members extends generally in the same direction and as a first one of the other pair of fixation members, and the second one of each pair of fixation members extends generally in the same direction as the second one of the other pair of fixation members, each fixation member terminating in footplate, and wherein the footplates of the first fixation members are spaced more closely together than footplates of the second fixation members.

Each and every feature described herein, and each and every combination of two or more of such features, is included within the scope of the present invention provided that the features included in such a combination are not mutually inconsistent.

These and other aspects and advantages of the present invention will become apparent in the following detailed description and claims, particularly when considered in conjunction with the accompanying drawings in which like parts bear like reference numerals.

### BRIEF DESCRIPTION OF THE DRAWINGS

FIG. 1 is a plan view of a first embodiment of an intraocular lens of the present invention having four substantially identical fixation members directly attached to interface portions extending outwardly from an optic; and

FIG. 2 is a plan view of a second embodiment of an intraocular lens of the present invention having two pairs of differently configured fixation members attached via a connecting bridge to flanges extending outward from an optic.

### DESCRIPTION OF THE PREFERRED EMBODIMENTS

The present invention provides anterior chamber intraocular lenses having flexible optic portions enabling folding, and relatively more rigid fixation members or haptics for sufficient fixation force. The lenses of the present invention include an interface portion attached to but out of the typically circular optic to which the fixation members attach. Each pair of fixation members attaches to the interface portion in a manner that sets up a line of force transmission through the interface portion that is outside the optic. In this manner, the optic is prevented from deformation upon compression of the fixation members, instead, the compressive forces are transmitted between the pair of fixation members and ultimately to the surrounding anterior chamber. The fixation members may be attached directly or via a bridge to the interface portions using conventional means, such as adhesives or interference fits.

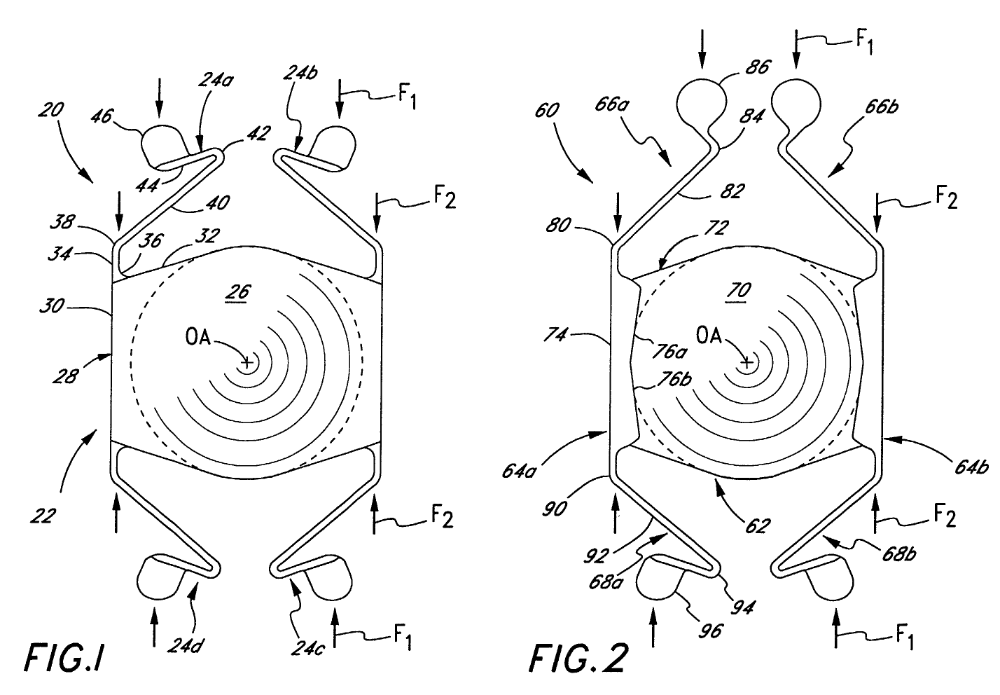

With reference to FIG. 1, a first embodiment of an intraocular lens 20 of the present invention includes an optic portion 22 and four fixation members 24a, 24b, 24c, 24d projecting outwardly therefrom. The optic portion 22 includes a typically circular optic 26 centered about an optical axis OA and illustrated in dashed line by a circle, and pair of outwardly extending flanges 28. The fixation members 24a, 24b, 24c, 24d are coupled to the optic 26 via the intermediary flanges 28, and thus the flanges comprise optic/fixation member interface portions.

Each flange 28 is desirably planar and extends generally radially outwardly from the optical axis OA to terminate in a straight edge 30. Desirably, the straight edge 30 of both flanges 28 are aligned in parallel. Each flange 28 further is defined by a pair of side edges 32, preferably straight, that extend from a port of tangency with the optic 26 and converge toward each other as shown. The included angle defined by each pair of converging side edges 32 is desirably between about 30-60 degrees, more preferably about 48 degrees.

The flanges 28 provide a line of compressive force transmission that does not intersect the optic 26 between each commonly attached pair of the fixation members 24a, 24b, 24c, 24d. In this regard, each fixation member 24a, 24b, 24c, 24d attaches to a side edge 32 of one of the flanges 28, and preferably directly across the flange 28 along a line that is parallel to the straight edge 30. Proximal ends 34 of either pair of fixation members 24 commonly attached to each flanges 28 extend in a co-linear fashion away from one another.

Each fixation member 24 comprises the proximal end 34 secured to the associated flange side edge 32 at a widened reinforcement shoulder 36. Moving outward on the fixation member, an inward bend 38 forms a transition between the relatively straight proximal end 34 and an elongated central portion 40. The term “inward” in this regard refers to the direction that each fixation member is bent with respect to the next closest fixation member. Therefore, the fixation member 24a includes a central portion 40 that is bent toward the fixation member 24b, and vice versa. Looking further along the fixation member from the central portion 40, a sharp bend 42 forms a transition between the central portion and an outer end 44. Each fixation member further includes a relatively rounded “pod” or footplate 46 designed to contact the iridio-corneal angle within the eye. The footplates 46 are wide and rounded to reduce potential for irritation and/or damage to the soft tissue of the iridio-corneal angle. The footplates 46 are substantially wider than the remainder of the fixation member, in particular the central portion 40 which must be relatively flexible. In the embodiment of FIG. 1, all four fixation members 24a, 24b, 24c, 24d are substantially identical.

At this point it is important to note that the term “fixation member” refers to the entire structure from the proximal end 34 to the footplate 46. In other words, each fixation member commences at a central location adjacent the optic and extends outward toward the iridio-corneal angle. Some designs of the prior art include more than one footplate along a member extending outward from the optic, but such designs should not be construed as more than one fixation member.

In use, compressive forces F1 are imparted to each of the footplates 46 by the surrounding iridio-corneal angle. Because of their rigidity, each fixation member 24a, 24b, 24c, 24d provides sufficient compressive retention force to maintain the optic portion 22 in position in the eye, and in particular to maintain the optical axis OA along the natural optical axis of the eye. Each fixation member 24a, 24b, 24c, 24d may flex somewhat to accommodate these compressive forces, and desirably flex about the inner bend 38, central portion 40, and outer bend 42.

Because of the advantageous use of the optic/fixation member interface portions (e.g., the flanges 28), the compressive forces imparted to the lens 20 are not transmitted through the optic 26. Therefore, any unwanted deformation from the compressive forces will not be transmitted to the relatively soft and foldable optic 26, and therefore will prevent distortion of any images transmitted therethrough. In this regard, the forces F1 are eventually transmitted to the proximal end 34 of each fixation member, as indicated by the force arrows F2. These resultant forces F2 for each pair of fixation members commonly attached to one of flanges 28 are aligned and tend to cancel each other out. That is, the material of the flange 28 between the point of attachment of the fixation members absorbs the resultant opposed forces F2. Desirably, therefore, the size and material of each flange 28 is sufficient to withstand excessive deformation, at least to the extent of preventing the deformation from affecting the optic 26.

It should be noted that the lenses of the present invention include at least two pairs of fixation members for more uniform lens support in the eye. That is, in contrast with tripod fixation member designs, the four fixation members provide a more uniform force distribution from the surrounding eye to the optic. In designs having only three fixation members, or only two with three footplates, a higher force is associated with the single footplate on one side of the optic. This may create problems with the iridio-corneal angle, or it may cause the lens to decenter. Furthermore, having two points of attachment on opposite sides of the optic as in the present invention is more desirable than just one. There is less tilt, better centration, and generally greater stability with the two opposed attachment points than with just one.

In a preferred embodiment, the fixation members 24a, 24b, 24c, 24d are made of a relatively rigid material, such as PMMA, suitable for maintaining position of the optic portion 22. The optic portion 22, and other hand, is made of flexible material suitable for folding within a Bartell-style inserter. For example, the optic portion 22 may be made of the material such as silicone or foldable acrylic. The fixation members 24a, 24b, 24c, 24d are attached to the respective flanges 28 by bonding or mating. A mated attachment typically refers to an interference fit. In this regard, the side edges 32 provide bonding or mating surfaces.

The resulting intraocular lens 20 can easily be folded about an axis that is perpendicular to the direction that the flanges 28 extend. That is, in the illustrated embodiment, the flanges 28 extend left and right, and the intraocular lens 20 is folded about a vertical axis. Although the fixation members 24a, 24b, 24c, 24d are relatively rigid, the space created between each two fixation members that are on opposite side of the fold axis permits the lens to be folded.

FIG. 2 illustrates a further intraocular lens 60 of the present invention having an optic portion 62, and pair of fixation systems 64a, 64b each having two fixation members 66, 68 associated therewith. More specifically, the left-hand fixation system 64a has an upper fixation member 66a and a lower fixation member 68a, while the right-hand fixation system 64b has an upper fixation member 66b and a lower fixation member 68b.

As in the embodiment of FIG. 1, the optic portion 62 includes a generally circular optic 70 circumscribed by a dashed line, and a pair of outwardly extending flanges 72. The flanges 72 are desirably configured the same as the flanges 28 described with respect to the earlier embodiment, such that they each have outwardly converging side edges terminating in a straight outer edge.

A bridge 74 extends between the upper and lower fixation members 66, 68 of each fixation system. The bridges 74 of each fixation system 64a, 64b attach directly to the outwardly extending flanges 72 using a suitable adhesive, or similar expedient. In one embodiment, a linear groove may be provided in each bridge 74 to receive the associated flange 72, and the assembly secured together with an interference fit or with the use of adhesives. Each bridge 74 includes an inner edge facing the optic 70 that is contoured to remain just outside of the circular outline of the optic. For example, and as illustrated, the inner edge may include two straight edge portions 76a, 76b that are angled at a central point generally toward the optic 70.

Each of the upper fixation members 66a, 66b extends directly away in a co-linear fashion from the associated lower fixation member 68a, 68b and turns inward at bend 80 to a central elongated portion 82. An outer bend 84 leads to an outer footplate 86. Each of the lower fixation members 68a, 68b turns inward at bend 90 to a central elongated portion 92, and includes a second bend 94 leading to an outer footplate 96.

As the first embodiment, external forces F1 are transmitted through the respective fixation members 66, 68 into forces F2 that are transmitted directly across each of the bridges 74. The bridges 74 thus absorb these opposed compressive forces and prevent them being imparted to the optic 70 to avoid distortion. Stated another way, each pair of upper and lower fixation members 66, 68, respectively, are attached to the optical portion 62 such that a direct line of force transmission is created therebetween outside of the optic 70.

It should be noted that the upper fixation members 66a, 66b are configured differently than the lower fixation members 68a, 68b. Specifically, the footplates 86 of the upper fixation members 66 are spaced more closely than the footplates 96 of the lower fixation members 68. The intraocular lens 60 is inserted in the eye such that the upper fixation members 66 are at the trailing end, and enter the eye last. The closely spaced footplates 86 are more easily placed into the anterior chamber at this trailing end position.

While this invention has been described with respect to various specific examples and embodiments, it is to be understood that the invention is not limited thereto and that it can be variously practiced within the scope of the following claims.

What is claimed is:

1. A foldable intraocular lens for implantation in the anterior chamber of an eye, the intraocular lens comprising:
an optic centered on an optical axis;
at least two pairs of fixation members each having a proximal end and a distal end adapted to engage the anterior chamber of an eye; and
at least two optic/fixation member interface portions to which the optic and the proximal ends of the fixation members attach, the interface portions each providing a line of compressive force transmission that does not intersect the optic between the proximal ends of each pair of fixation members.

2. The intraocular lens of claim 1, wherein the optic is circular and the interface portions extend outward from the optic to form planar flanges.

3. The intraocular lens of claim 2, wherein the two flanges extending outward from opposite sides of the optic.

4. The intraocular lens of claim 3, wherein each flange is defined by two side edges that project generally tangentially from the circular optic and converge toward each other in an outward direction from the optic, and an outer straight edge.

5. The intraocular lens of claim 4, wherein the line of compressive force transmission between the proximal ends of each pair of fixation members extends parallel to and adjacent the outer straight edge of the respective flange.

6. The intraocular lens of claim 3, wherein the proximal ends of each pair of fixation members are co-linear.

7. The intraocular lens of claim 6, wherein each fixation member includes a central portion that converges inward toward a second fixation member, and wherein the distal end of each fixation member terminates in a footplate that is wider than the central portion.

8. The intraocular lens of claim 1, wherein the proximal ends of each pair of fixation members are co-linear.

9. The intraocular lens of claim 8, wherein the distal end of each fixation member terminates in a footplate that is wider than the proximal end.

10. The intraocular lens of claim 1, wherein each pair of fixation members is formed of a single element, the fixation members being connected by a bridge that attaches to the interface portion.

11. A foldable intraocular lens for implantation in the anterior chamber of an eye, the intraocular lens comprising:
an optic centered on an optical axis;
a pair of planar flanges extending in opposite directions from the optic; and
two pairs of fixation members each having a proximal end attached to one of flanges and a distal end adapted to engage the anterior chamber of an eye, the attachment of each pair of fixation members to the flanges providing a line of compressive force transmission that does not intersect the optic between the proximal ends of the two fixation members in the pair.

12. The intraocular lens of claim 11, wherein the optic is circular and each flange is defined by two side edges that project generally tangentially from the circular optic and converge toward each other in an outward direction from the optic, and an outer straight edge.

13. The intraocular lens of claim 12, wherein the line of compressive force transmission between the proximal ends of each pair of fixation members extends parallel to and adjacent the outer straight edge of the respective flange.

14. The intraocular lens of claim 11, wherein the proximal ends of each pair of fixation members are co-linear.

15. The intraocular lens of claim 14, wherein each fixation member includes a central portion that converges inward toward a second fixation member, and wherein the distal end of each fixation member terminates in a footplate that is wider than the central portion.

16. The intraocular lens of claim 11, wherein each pair of fixation members are formed of a single element and connected by a bridge that attaches to the flange.

17. The intraocular lens of claim 16, wherein the optic is circular and each flange is defined by two side edges that project generally tangentially from the circular optic and converge toward each other in an outward direction from the optic, and an outer straight edge wherein each bridge attaches to the outer straight edge of the respective flange.

18. The intraocular lens of claim 17, wherein each bridge includes a groove into which the outer straight edge of the respective flange inserts.

19. The intraocular lens of claim 18, wherein the outer straight edge of each flange inserts into the groove of the respective bridge a depth that places an inner edge of the bridge generally on a tangent to the circular optic.

20. The intraocular lens of claim 19, wherein the inner edge of each bridge includes a central corner that is recessed away from the optical axis and pair of half-edges diverging outwardly from the central corner and angled generally in the direction of the optic.

21. The intraocular lens of claim 11, wherein all four fixation members are identical in configuration.

22. The intraocular lens of claim 11, wherein a first one of each pair of fixation members extends generally in the same direction and as a first one of the other pair of fixation members, and the second one of each pair of fixation members extends generally in the same direction as the second one of the other pair of fixation members, each fixation member terminating in footplate, and wherein the footplates of the first fixation members are spaced more closely together than footplates of the second fixation members.
